(19) United States
(12) Patent Application Publication
(10) Pub. No.: US 2019/0059870 A1
(43) Pub. Date: Feb. 28, 2019

(54) SURGICAL RETRACTION DEVICE AND PROCEDURE

(71) Applicant: University of Leeds, Leeds (GB)

(72) Inventors: David G. Jayne, Leeds (GB); Peter Culmer, Leeds (GB); Mohammed Sanami, Leeds (GB); Richard Hall, Ripon (GB); Timothy Flood, Ripon (GB)

(73) Assignee: University of Leeds, Leeds (GB)

(21) Appl. No.: 16/074,980

(22) PCT Filed: Jan. 20, 2017
(86) PCT No.: PCT/GB2017/050140

§ 371 Date: Aug. 2, 2018

(30) Foreign Application Priority Data
Feb. 2, 2016 (GB) 1601858.2

(51) Int. Cl. A61B 17/02

(52) U.S. Cl. CPC A61B 17/0218; A61B 2017/00991; A61B 17/0206; A61B 17/0281

(57) ABSTRACT

The present invention provides a retraction device comprising, a shaft, a plurality of arms supported by the shaft, the arms being configured to be moveable between a first orientation in which the device assumes an insertion profile for passage of the device through a surgical incision, and a second orientation in which the device assumes a retraction profile. At least one of the plurality of the arms is adapted to be adjustable between a first length and a second length.

### FIELD OF THE INVENTION

The invention is concerned with a retraction device for use in retracting objects, in particular organs and tissue during surgical procedures. A kit including the retraction device and a method for use of the device is also described.

### BACKGROUND OF THE INVENTION

The advent of laparoscopic surgery, which is also known as minimally invasive surgery (MIS) or key-hole surgery has revolutionised clinical practice and has proven benefits in terms of less postoperative pain, shorter hospital stays, quicker return to normal function, fewer wound complications and reduced external scarring. The continued implementation of this technique has resulted in predictions that 60-90% of all general surgical procedures will be performed using this technology in the future.

However, the application of MIS presents significant challenges due to the fact that the surgeon's hands are replaced by laparoscopic/endoscopic instruments which are manipulated from outside the body cavity and are typically fairly simplistic devices which further limit dexterous or precise movements. Additionally, navigation is often difficult, since the surgeon's view is provided by a 2D assistant-controlled. camera.

A key surgical principle, in the context of either open surgery or MIS, is the ability to adequately retract tissues and organs so as to expose the operative field, and to facilitate dissection. This includes the principle of “triangulation” in which opposing forces are applied to open up normal tissue planes.

In MIS, triangulation is made difficult by the restrictive environment of the closed body cavity and the lack of suitable instrumentation. Current retractors for MIS are relatively primitive, consisting of ratcheted devices of varying shapes and sizes. Although some progress has been made in the development of “bending” graspers, significant further advances are necessary in order to improve capability if retraction is to be as good as in open surgery. Most particularly, an improved device/tissue interface is required to reduce the risk of iatrogenic tissue injury, and it is especially desirable that the retraction device should be “hands-free”, thereby enabling a surgeon to concentrate on the operation in progress without dependency on an assistant.

In open surgery, systems which deploy several retractors on a stabilising framework are often used to maximise exposure. An example of such a system is the Omnitract® surgical retractor, wherein a scaffold is anchored to the operating table, and several retracting arms can be attached to the scaffold in order to apply multi-directional retraction to the abdominal wound/tissues. A suitable MIS retraction system might employ a similar strategy.

Various devices are described in the prior art for use in surgical procedures. Many devices require external fixing scaffolding around the operating table so that they can be fixed to it during the duration of the procedure. For example, U.S. Pat. No. 5,704,900 discloses a surgical retraction device in which an expandable retraction means is connected, via a flexible attachment means, to the operating table. This can occupy valuable space and hinder the surgeon in his performance of the surgery. The flexible attachment means are typically reusable and therefore require sterilization and maintenance, which have financial implications.

A further problem with prior art devices, such as the device disclosed in US-A-2007/0032704, is that the retraction arms have a fixed length. This can result in the insufficient retraction of organs and tissue away from the actual surgical site, that is, the site that requires therapeutic and/or surgical intervention. The occlusion of the actual site of surgery poses risks that non-targeted tissue will become damaged during the procedure. In particular in colorectal cancer procedures there are a significant number of conversions to open surgery as a result of operative difficulties.

### SUMMARY OF THE INVENTION

According to an aspect of the invention, there is provided a retraction device comprising:

a shaft,

a plurality of arms supported by the shaft and being configured to be moveable between a first orientation in which the device assumes an insertion profile for passage of the device through a surgical incision, and a second orientation in which the device assumes a retraction profile, and in which at least one of the plurality of the arms is adapted to be adjustable between a first length and a second length.

According to a further aspect of the invention there is provided a retraction device comprising:

a shaft,

a plurality of arms supported by the shaft, each arm being configured to be moveable between a first orientation in which the device assumes an insertion profile for passage of the device through a surgical incision, and a second orientation in which the device assumes a retraction profile, and

a clamp mounted and arranged on the shaft in a manner such that a contact surface of the clamp contacts an exterior surface of the skin of the subject.

Following insertion of the retraction device through a surgical incision into a body cavity, the surgeon will deploy the device from the “closed” insertion profile such that it assumes the “open” retraction profile.

When the device is in the retraction profile, a proximal end of the shaft of the device will remain on the outside of the surgical incision. The surgeon can then position a clamp at a suitable position along the exposed portion of the shaft so that a contact surface of the clamp is brought into contact with the skin of the subject. In this arrangement a body wall, for example the abdominal wall, is sandwiched between the contact surface of the clamp and the upper surface of the plurality of arms. This ensures that the scaffold, formed by the plurality of expanded arms inside the body cavity, is stable both in terms of position and orientation. It is advantageous that when a tissue or an organ is pulled towards the scaffold and attached to it, the position and orientation of the scaffold is not altered, as this could compromise the surgical access. Furthermore, it is desirable that the scaffold/platform remains as close to the interior surface of the body wall as possible, thus providing the surgeon with maximum available space within which to work. In minimally-invasive procedures in which an “artificial” pneumoperitoneum is intentionally generated within a body cavity by insufflation with a gas (e.g., CO₂), the clamp also provides an extra seal at the surgical incision that helps to minimise the escape of the gas.

This stable arrangement of the retraction device also eliminates the need for the device to be connected to a rigid support means. However, it is envisaged that in some circumstances a surgeon may decide to connect the retraction device to a rigid support means, such as an operating table, if additional support is required. Indeed, some surgeons may have a preference for using this approach.

In some constructions the clamp is pre-mounted on the device. The surgeon can move the clamp vertically along the length of the exposed portion of the shaft, as required, in order for the contact surface of the clamp to be brought into contact with the exterior surface of the skin.

In other constructions the clamp is removably attachable to the shaft of the device. The clamp can be attached to the device either when the device is in the insertion profile or the retraction profile.

In some constructions the clamp can be slidably mounted on the shaft. The shaft can include a guide channel dimensioned to fit around at least part of the circumference of the shaft. The guide channel guides movement of the clamp along the length of the shaft. The guide channel can be provided as a bore that extends through the clamp. This bore can be formed during the manufacture of the device or can be formed during the use of the device. For example, in some constructions, the clamp can include a first member having a first guide channel and a second member having a counterpart second guide channel, the bore being formed by the first channel and the second channel.

The first member and second member can be pivotally connected. In this construction the surgeon can wrap the first and second members around the shaft so that the shaft is received within the bore formed by the first and second channel The first and second members can be retained in the closed configuration about the shaft by the provision of a locking mechanism. For example, a snap-fit closure, or a latch mechanism.

In some constructions the clamp includes a flange extending radially outwardly therefrom. A contact surface of the flange rests on the exterior surface of the skin of the subject. The flange can be annular.

It is envisaged that a plurality of clamps can be provided, with each clamp being provided with a different size of flange. This allows the surgeon to select a clamp that is considered will provide the most stable arrangement of the retraction device.

In some constructions each arm of the plurality of arms is independently moveable from the first configuration to the second configuration. In other constructions, each arm of the plurality of arms moves simultaneously with each of the other arms when the device is deployed from the insertion profile to the retraction profile. The arms can be moved from the first orientation to the second orientation by mechanical means or by insufflation, or a combination thereof.

In preferred constructions, in the second orientation the angle between each arm and the longitudinal axis of the shaft is at least about 70°. For example, at least about 80° or at least about 90°. Each arm can be reversibly locked in this angular position. The angle that one arm assumes relative to the longitudinal axis of the shaft can be the same as the angle that each other arm of the plurality of arms assumes relative to the longitudinal axis. In other constructions, the angle that one arm assumes relative to the longitudinal axis of the shaft can be different from the angle that another arm of the plurality of arms assumes relative to the longitudinal axis.

In some constructions, the retraction device has two arms which are pivotally opened and closed in the manner of a folding fan. This is similar in operation to a speculum. The angle between the arms in the second orientation can be at least about 50°. For example, at least about 60°, or at least 70°, or at least 80°. In other constructions, there are at least three arms.

In some constructions the shaft has a perimeter and the plurality of arms are arranged about the perimeter such that in the insertion profile a longitudinal edge of a first arm is in contact with a longitudinal edge of a neighbouring arm. This provides the retraction device with a smooth external profile. This is advantageous because the smooth external profile minimises the risk of any tissue becoming snagged on or in the device during insertion through the surgical incision. A magnetic material can be provided on the inner surface of each of the arms to hold the arms together in a snug relationship in the insertion profile. Other fastening means are envisaged, for example, male-female connections.

In some constructions the plurality of arms are arranged about substantially all of the perimeter of the shaft and extend therefrom. In other constructions, the plurality of arms are spaced apart around the perimeter of the shaft.

In some constructions at least one of the plurality of arms functions as a “retraction” arm and at least one other arm functions as a “stabilisation” arm.

The retraction arm is provided with a restraint that is capable of pushing or pulling, and subsequently restraining, a tissue or organ within the surgical site. Such restraints are known to a skilled artisan and non-limiting examples include: clips, hooks, bands, ties, sutures, slings, loops, sockets, suction pads, Velcro, nets and spatulas.

The stabilisation arm is designed to help stabilise the retraction device. It will normally be positioned outside of the surgical field. The stabilisation arm is generally shorter and wider than the retraction arm(s).

In some constructions all of the plurality of arms function as retraction arms.

In some constructions the shaft of the retraction device is a substantially circular cylinder. It is envisaged that in some constructions, a first arm extends from at least part of a first quadrant of the shaft and a second arm extends from at least part of a second quadrant of the shaft. The first arm functioning as a retraction arm and the second arm functioning as a stabilisation arm.

In other constructions a first arm extends from at least part of a first quadrant of the circular shaft, a second arm extends from at least part of a second quadrant of the shaft and a third arm extends from at least part of a third quadrant of the shaft. The first arm and second arms functioning as retraction arms and the third arm functioning as a stabilisation arm.

In some other constructions an arm extends from at least part of each quadrant of the circular shaft. That is to say, there are four arms distributed about the perimeter of the shaft. Advantageously, each longitudinal edge an arm is in contact with a longitudinal edge of a neighbouring arm. This enables the device to form a smooth external profile during insertion. However, it is also contemplated that each arm may be spaced apart from at least one neighbouring arm. This leads to a gap between at least two neighbouring arms. In the described construction, at least three of the arms function as retraction arms, whilst the fourth arm functions as a stabilisation arm.

In some constructions at least one of the plurality of arms is removably attachable to the shaft. This provides the surgeon with the flexibility to remove at least one of the arms if it is considered that the presence of the arm will hinder the surgical procedure.

In some constructions at least one of the plurality of arms is moveable about the perimeter of the shaft. This provides the surgeon with the flexibility to strategically move at least one of the arms to a position that is considered to provide the optimal position for the retraction of a tissue or an organ. This rearrangement of the arms could be undertaken either pre- or intra-operatively.

In some constructions each arm is pivotally connected to the shaft. This connection is generally provided at or near the distal end of the shaft.

In other constructions the plurality of arms comprise mechanical members slidably attached to the shaft and interlinked by a series of interlinking members, typically linking pins, such that they form an umbrella arrangement in the retraction profile. In some other constructions of the device, each of the plurality of arms comprises a radially-orientated channel configured for receipt of an inflation fluid. Insufflation of each arm causes each arm to move from the first orientation to the second orientation.

It is particularly advantageous that at least one of the plurality of arms is adapted to be adjustable between a first length and a second length. This allows the surgeon to manipulate the device to provide sufficient retraction of tissues and organs within the surgical site, so that the target tissue is readily accessible. The ability to adjust the length of the arm includes being able to shorten or lengthen the length of the arm, as required. Preferably, the surgeon can adjust the length of the arm once the retraction device has been inserted into surgical site. This ensures that the insertion profile is not overly long, thereby reducing the risk that underlying tissue becomes inadvertently damaged during the insertion process.

The adjustability of arm length can be achieved by one arm being telescopic. In one particular construction, the arm includes a first portion that connects to the shaft and a second portion that is slidably connected to the first portion. Tensionable cord extends along the length of the shaft and along the first and second portions of the arm.

When the arm is configured to have its first length, the cord is not held under tension and the proximal end of the second portion (i.e. the end nearest to the first portion) overlaps with the distal end of the first portion (i.e. the end nearest to the second portion). In some constructions, the proximal end of the second portion lies underneath the first portion. When the surgeon puts the cord under tension, by applying a pulling action to the cord, the second portion of the arm is caused to slide in a direction away from the first portion, thereby extending the overall length of the arm. In some constructions, when the cord is fully tensioned an overlap between the proximal end of the second portion and the first portion will remain. In some other constructions, when the cord is fully tensioned the second portion of the arm becomes fully aligned with the first portion.

By applying tension to the cord, a surgeon is able to extend the arm to any desirable length, up to a maximum length. In some constructions the arm is lengthened such that the second length is at least about 1.5 times longer than the first length. For example at least about 1.6, about 1.7, about 1.8, about 1.9 or about 2.0 times longer than the first length. Of course, the surgeon can select to extend the arm to a length that is less than about 1.5 times longer than the first length.

In some constructions the length of each of the retraction arms is adjustable. Preferably, the length of each of the retraction arms is independently adjustable. It is envisaged although the stabilisation arm (if present) is generally positioned outside of the surgical field, its length may also be adjustable.

In some constructions each arm consists of or comprises a metal. It is however envisaged that each arm could instead comprise a polymeric material. Advantageously, at least one magnetic anchoring point for a magnetic tensile restraint, for example a magnetic clip, is provided on each arm. The use of magnetic tensile restraints is particularly advantageous because any lost tensile restraints (i.e. restraints that have been dropped within the body cavity) can be retrieved using a magnet.

In the constructions in which each of the plurality of arms consists of or comprises a metal, the metal is magnetic. More specifically the metal is ferromagnetic.

In the constructions in which each of the plurality of arms comprises a polymeric material, the at least one magnetic anchoring point is provided in the form of a magnetic area positioned on each arm. This at least one magnetic area can take the form of a magnet that is disposed at a position along the length of each arm. In other constructions, at least two magnetic areas are provided on each arm, thereby providing multiple points for magnetic anchorage.

It is envisaged that the magnet(s) can be provided, for example, as a strip(s), disc(s) or bead(s) on or within each arm. Preferably, the magnet(s) is/are embedded within the polymeric material in order to avoid direct contact with tissue.

In some constructions, the magnet is an electromagnet. In this construction, the magnetic field can be conveniently controlled, such that its magnitude may be increased or decreased, or it may be switched off completely, according to requirements.

It is also envisaged that in some constructions each arm may comprise at least one channel that comprises at least one ferrofluid.

In some constructions, the retraction device further comprises a slidably moveable member capable of movement relative to the shaft,

in which relative movement of the slidably moveable member in a first direction radially collapses the arms causing the retraction device to assume the insertion profile, and

in which relative movement of the slidably moveable member in a second direction radially expands the arms causing the retraction device to assume the retraction profile.

The slidably moveable member can be, for example, a plunger that is provided at the top of the shaft, in a similar manner to a syringe. The downward, or conversely upward, movement of the plunger results in the movement of each of the plurality of arms from the first orientation to the second orientation.

According to another aspect of the invention, there is provided a kit for use in a surgical procedure, the kit comprising a retraction device as provided in the first or second aspects of the invention, and at least one of the group consisting of:
i) an access means through which the retraction device may be passed;
ii) a flexible attachment means configured to secure the retraction device to a rigid support means;
iii) a clamp configured for mounting on the shaft of the retraction device;
iv) a restraining means for attachment to at least one of the plurality of arms of the device, the restraining means being configured for restraining a tissue or organ;
v) a restraining means deployment tool.

When performing some surgical procedures, a surgeon may choose to use an access means through which the retraction device is inserted, rather than directly inserting the retraction device through the surgical incision. The access means can be an access port through which a surgical device, such as a standard laparoscopic camera, may be passed. Typically, the access port has a diameter in the region of 5-20 mm, more preferably 10-15 mm and, most preferably, about 10-12 mm Suitable devices for use as access ports are laparoscopic trocars or ports, which may either be of conventional design or may optionally be of the SILS (single-incision) type.

Conventional trocars are well known to those skilled in the art and are shaped like pen and have sharp triangular points. Laparoscopic trocars play an important role in laparoscopic surgery, and instruments such as scissors and graspers may be introduced using surgical trocars. Modern-day trocars have eliminated the need for large abdominal incisions, which has contributed greatly to improved patient care. Surgical trocars are typically single-patient use and disposable and are often entirely without a blade and incision made with a scalpel, or have a linear sharp blade designed to minimize the force required to insert the instrument, making them safer to use when introducing laparoscopic ports and helping to avoid inadvertent tissue damage. Wound closure and healing is much faster as these incisions are smaller and tissue damage to the abdominal wall is decreased.

SILS devices are designed to give surgeons the ability to use multiple instruments with maximal maneuverability through adjustable cannulas within low-profile malleable ports and typically comprise flexible laparoscopic ports that can accommodate up to three instruments through a single incision.

A flexible attachment means, if provided as part of the kit, preferably comprises a plurality of interlocking rigid members comprising a hinge mechanism located externally to the subject. The flexible attachment means is configured to secure the retraction device to a rigid support means, such as an operating table.

In constructions in which the clamp is to be assembled onto the shaft of the device by the surgeon, a kit comprises the retraction device and at least one clamp is provided.

If a tensile restraining means is provided as part of a kit this may consist of or comprise a magnetic material. This enables the restraining means to be magnetically attached to the at least one magnetic anchoring point provided on at least one arm of the retraction device. The restraining means can be in the form of a magnetic clip.

According to yet a further aspect of the invention, there is provided a method for the use of a retraction device during a surgery procedure, the method comprising the steps of:
i) providing a retraction device according to the first or second aspects of the invention;
ii) inserting the retraction device into a body cavity so that a proximal end of a shaft of the device is external to said cavity;
iii) deploying the retraction device into its retraction profile.

The method can further comprise the steps of;
iv) magnetising tissue to be retracted; and
v) attaching the magnetised tissue to the retraction device.

Step iv) may be performed prior to step v). Alternatively, step v) may be performed prior to step iv).

The means for magnetising the tissue to be retracted can comprise injecting ferrofluids into the tissue, or affixing magnetic moieties to the tissue. The said magnetic moieties can be affixed to the tissue by clips.

The surgical procedure can be minimally invasive surgery of the abdomen or pelvis, conventionally referred to as laparoscopic surgery. Laparoscopy is most commonly used in the fields of gynaecology, gastroenterology and urology. Non-limiting examples of laparoscopic surgery include: cholecystectomy, colectomy, myomectomy, hysterectomy and cystectomy. The surgical procedure can be minimally invasive surgery of the plural and thoracic cavities, conventionally referred to as mediastinoscopy and thoracoscopy.

### BRIEF DESCRIPTION OF THE DRAWINGS

Constructions of the invention are further described herein with reference to the accompanying drawings in which:

FIG. 1 is a side view of the retraction device.

FIG. 2 is an end view of the distal end of the retraction device.

FIG. 3 shows the retraction device in the insertion profile as it is being inserted through the surgical incision.

FIG. 4 shows a detailed views of the clamp.

FIG. 5 shows assembly of the clamp onto the shaft of the retraction device.

FIG. 6 shows the clamp in its final position on the retraction device.

FIG. 7 shows the mechanism for the deployment of the retraction device from the insertion profile to the retraction profile.

FIG. 8 is a schematic of the retraction device in the retraction profile within a body cavity.

FIG. 9 shows the mechanism for extending the length of the arms of the retraction device.

FIG. 10 is a schematic of the retraction device shown in FIG. 9 within a body cavity.

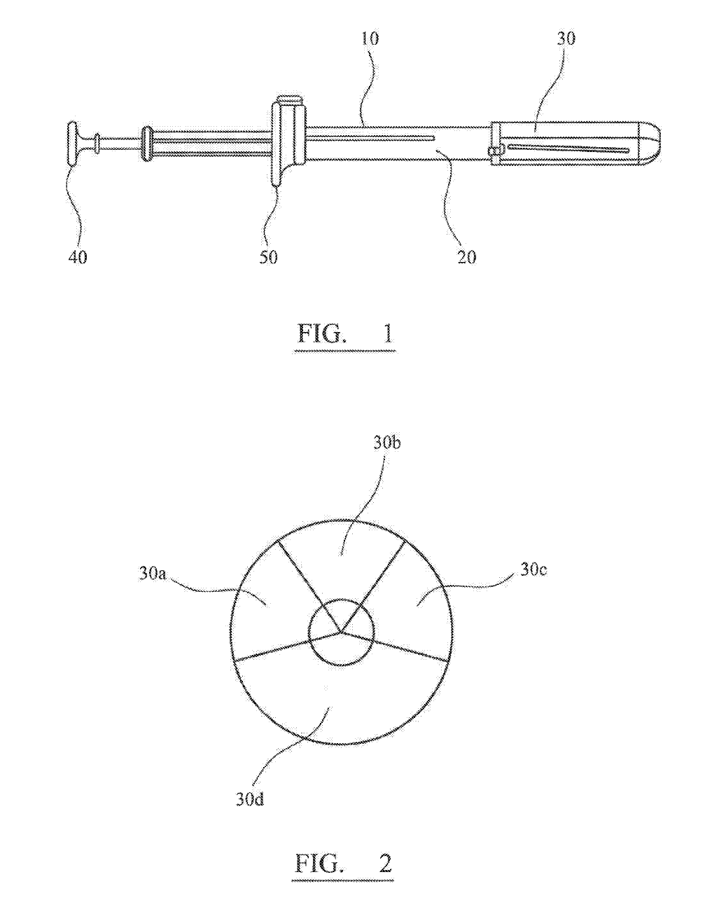

FIG. 1 shows a side view of the retraction device 10. The device is deployed to/from an insufflated abdominal cavity through a small incision (similar in size to that made by a 12 mm laparoscopic camera port). This minimises access trauma. The device is designed to be located at the central laparoscopic access point, typically the umbilicus. The device is inserted/removed from the incision in the same way as a conventional laparoscopic port or trocar. The device may be used in conjunction with an access means, such as a trocar, but this is not necessary. The device includes a shaft 20, a plurality of arms 30 located at or near the distal end of the shaft, a plunger 40 located at the opposing proximal end and a collar 50 positioned about the shaft.

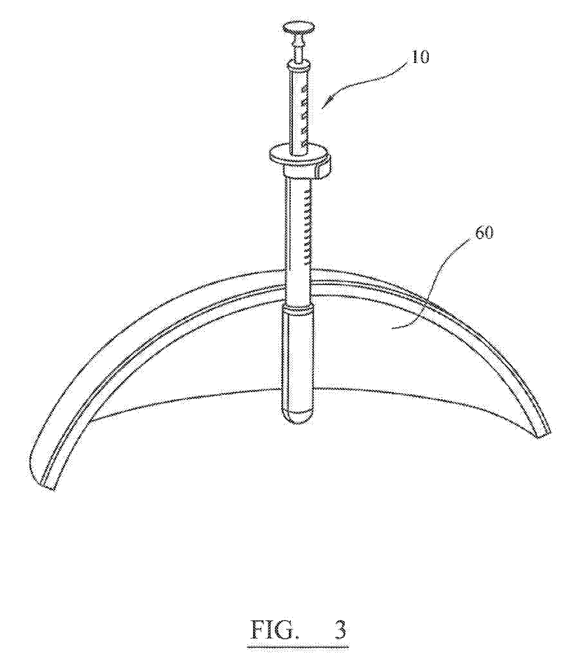

An end view of the retraction device 10 is shown in FIG. 2. The illustrated device includes three retraction arms 30a, 30b and 30c and one stabilisation arm 30d. The longitudinal edges of each of the arms are in contact with the longitudinal edges of the neighbouring arms. This minimises the cross-sectional size of the device and provides a smooth insertion profile. The proximal end of each arm has a tapered curve. This results in the distal-most end of each arm converging to a central point. This shaping also facilitates the insertion of the device through a surgical incision.

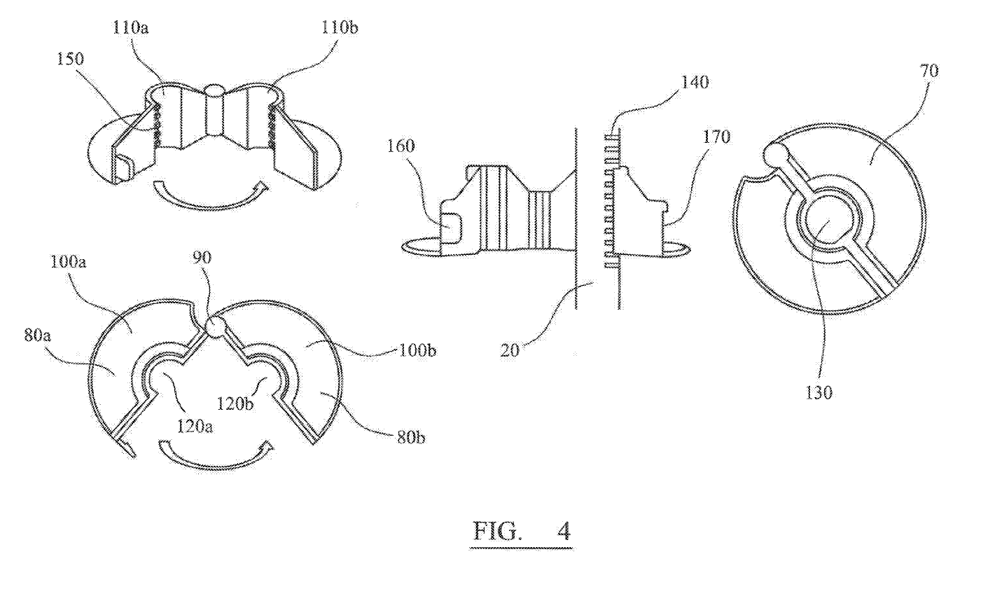

FIG. 3 is a schematic of the retraction device partly inserted through a surgical incision within the abdominal wall 60. The abdominal wall is shown in cut-away. The retraction device is shown in the “insertion” profile. Whilst the diameter of the device in the insertion profile is variable, it is constrained by the size of the surgical incision. The device must fit snuggly within the surgical incision. In the illustrated embodiment the diameter of the device is about 19 mm.

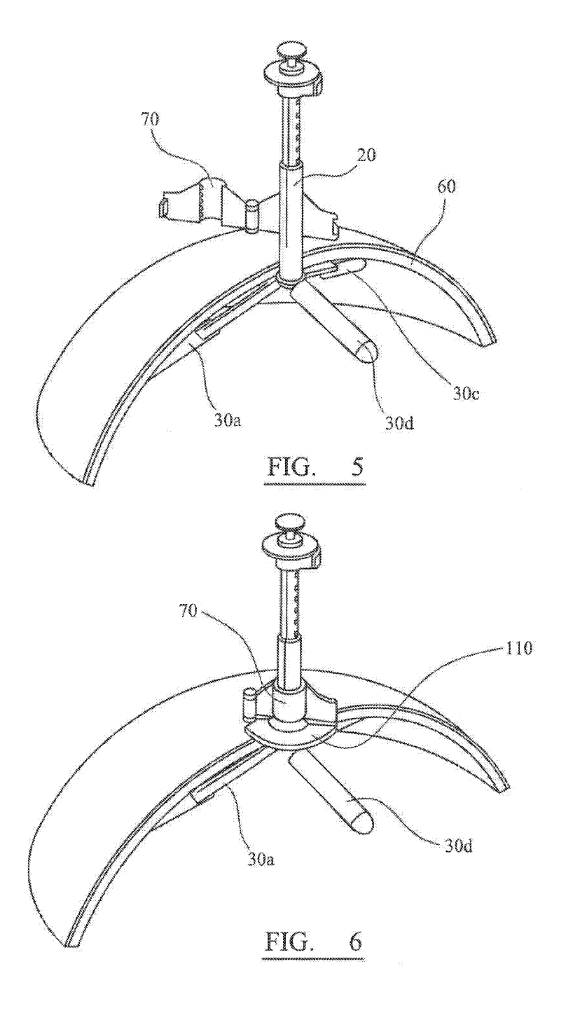

FIG. 4 shows the clamp 70 which is used to support the retraction device within the abdominal cavity. The use of the clamp obviates the need for the retraction device to be connected to a rigid support means, such as an operating table. The clamp is in the form of a collar that the surgeon positions about the shaft 20 of the device.

The clamp 70 comprises a first member 80a and a second member 80b that are pivotally connected via a hinge 90. Each member includes a semi-circular shaped flange 100a, 100b. An upstanding curved wall 110a, 110b is provided on the straight edge of the semi-circular flange. Each curved wall 110a, 110b provides a semi-circular guide channel 120a, 120b on each member. The guide channels 120a, 120b are located in corresponding positions on the first and second members so that when the two members are pivotally brought together into a closed relationship the guide channels 120a, 120b form a bore 130. The depth of the guide channels 120a, 120b is selected such that the final diameter of the bore 130 is only slightly larger than diameter of the shaft 20.

When the first and second members 80a, 80b are in the closed positioned the semi-circular flanges 100a, 100b form an annular flange.

The shaft 20 is provided along its length with a plurality of horizontally orientated grooves 140. Teeth 150 arranged on one end of each curved wall 110a, 110b are engagable with the grooves 140. This improves the attachment of the clamp to the shaft.

The clamp 70 is also secured in place about the shaft by a tab 160 provided on the straight edge of the first member 80a being snap-fitted into a slot 170 provided on a straight edge of the second member 80b.

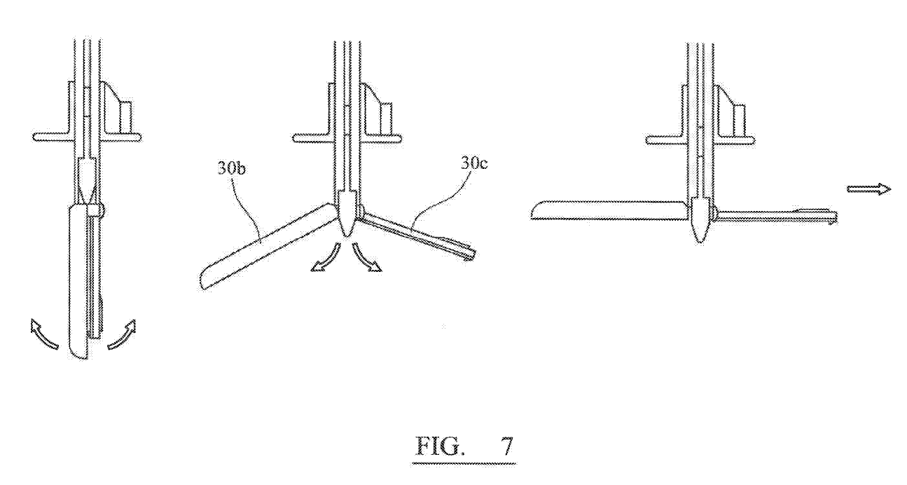

FIG. 5 illustrates the clamp 70 being positioned about the shaft 20 of the retraction device. The retraction device is shown in the “retraction” profile, with the plurality of arms in the second orientation. The abdominal wall 60 (shown in cut-away) is sandwiched between the lower surface of the annular flange, and the upper surfaces of each of the retraction arms 30a-30c. The clamp is shown in the closed position in FIG. 6. The clamp is positioned along the longitudinal axis of the shaft 20 so that the lower surface of the annular flange (formed by semi-circular flanges 110a and 110b) is in contact with the exterior surface of the skin of the patient. In this arrangement the clamp 70 can apply a compressive force downwardly onto the skin, thereby providing a sealing function. This helps to prevent gas leaking from the insufflated abdominal cavity.

### Deployment of the Device

The deployment of the device from the “insertion profile” to the “retraction profile” is described below as a two-step process. The first step is referred to as the “arm opening” step. The second step is referred to as the “arm lengthening step”. The second step is optional.

### First Step Of Device Deployment—the “Arm Opening” Step.

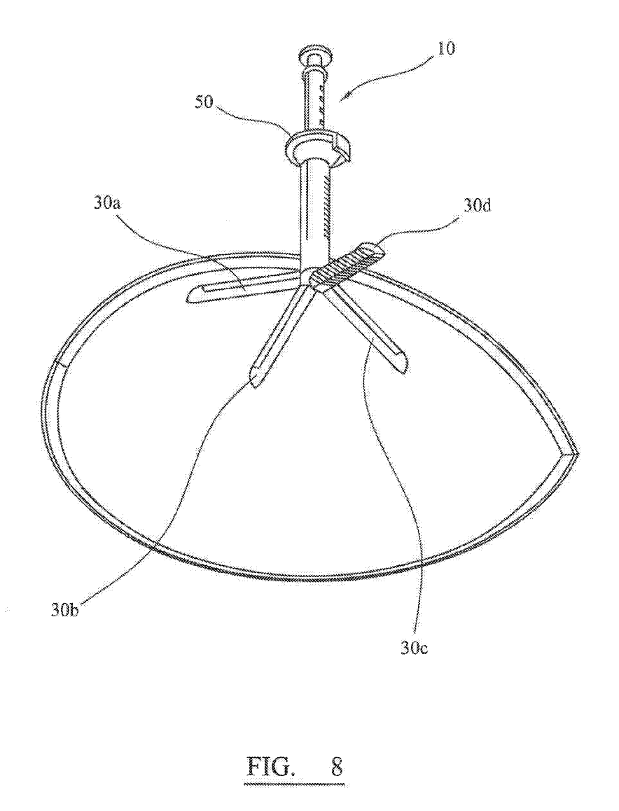

In this step the retraction arms 30a-30c and the stabilisation arm 30d are moved to their second orientation in which they become angled (Θ>0°) relative to the longitudinal axis shaft 20. This mechanism for moving the arms is illustrated in FIG. 7. The surgeon depresses the plunger 40 which in turn moves a cam-like mechanism past each arm.

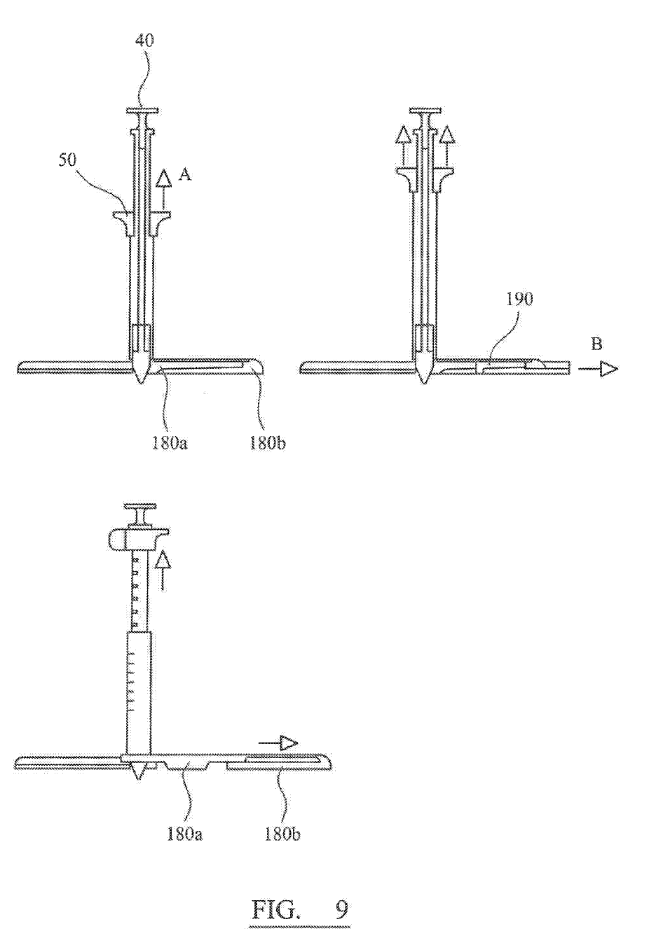

FIG. 8 illustrates the arms 30a-30d in the second orientation, in which the arms are locked at an angle of about 90° with respect to the longitudinal axis of the shaft 20. In this second orientation the upper surface of the arms lie against the inner surface of the abdominal wall. The angle between the arms 30a and 30b is about 70° and the angle between the arms 30b and 30c is about 70°. It is contemplated that the angle between the arms 30a and 30b and the angle between the arms 30b and 30c can be lesser or greater than about 70°. It is also contemplated that the angle between the arms 30a and 30b can be different from the angle between the arms 30b and 30c.

### Second Step of Device Deployment—the “Arm Lengthening” Step.

FIG. 9 illustrates the mechanism for extending the length of the retraction arms 30a-30c. In the device illustrated the stabilisation arm 30d is not extendable, but it is envisaged that it could be in other constructions. Each retraction arm includes a first portion 180a that is pivotally connected to the shaft 20 and a second portion 180b that is slidably connected to the first portion. A tensionable cord 190 extends along the length of the shaft and along the first and second portions of the arm. When the arm is configured to have its first length, the cord is not held under tension and the proximal end of the second portion lies underneath the first portion. When the surgeon puts the cord under tension, by sliding the collar 50 upwardly along the shaft 20 towards the plunger 40 (as indicated by the arrow A), the second portion 180b of the arm is caused to slide in a direction away from the first portion 180a (as indicated by Arrow B), thereby extending the overall length of the arm.

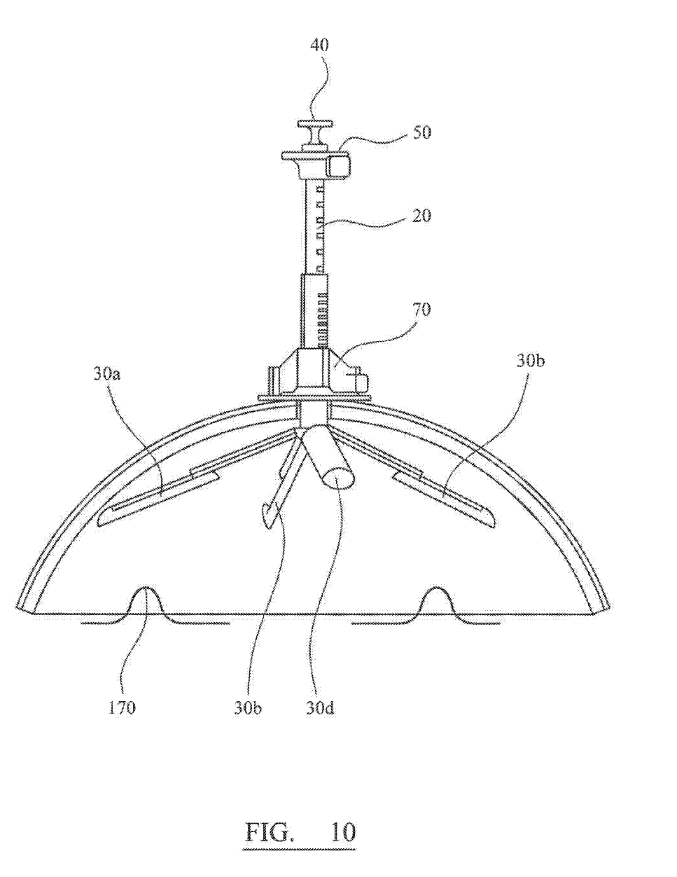

FIG. 10 illustrates the device with fully extended arms 30a-30c. Retraction of tissues is achieved using a system of tensile restraints 170 which are composed of a clip to grasp the desired tissue or organ which in turn is anchored to the lower surface of the retraction arms 30a-30c using mechanical fixings. Suitable mechanical fixings, include magnets, hooks or loops provided on the clip. Optionally, a compliant cord element joins the clip to the retraction arm.

After the completion of the surgical procedure the device 10 is removed from the abdominal cavity by reversing the deployment steps 1 and 2. The device assumes the original insertion profile, and can be removed from the abdominal cavity, once the clamp has been moved out of contact with the exterior surface of the skin of the patient.

It is envisaged that the reversal of the deployment steps can be performed in either order. That is to say, the arms can be shortened and then moved to the first orientation, or the arms can be moved to the first orientation and then shortened.

For example, the surgeon can slide the collar 50 downwardly along the shaft 20 to shorten the length of the arms 30a-30c. The plunger 40 is then moved upwardly to move the arms 30a-30d from the second orientation into the first orientation.

1. A retraction device comprising:
a shaft,
a plurality of arms supported by the shaft and being configured to be moveable between a first orientation in which the device assumes an insertion profile for passage of the device through a surgical incision, and a second orientation in which the device assumes a retraction profile, and in which at least one of the plurality of the arms is adapted to be adjustable between a first length and a second length.

2. The retraction device as in claim 1, in which the at least one arm is a telescopic arm.

3. The retraction device as in claim 1, in which the second length is longer than the first length.

4. The retraction device as in claim 3, in which the second length is at least 1.5 times longer than the first length.

5. The retraction device as in claim 1, in which the device further comprises a slidably moveable member capable of movement relative to the shaft,
in which relative movement of the slidably moveable member in a first direction radially collapses the arms causing the retraction device to assume the insertion profile, and
in which relative movement of the slidably moveable member in a second direction radially expands the arms causing the retraction device to assume the retraction profile.

6. The retraction device as in claim 1, in which the device further comprises a clamp mounted and arranged on the shaft in a manner such that a contact surface of the clamp contacts an exterior surface of the skin of the subject.

7. The retraction device claim 6, in which the clamp includes a flange extending radially from a distal end thereof, a lower surface of said flange making contact with the surface of the skin.

8. The retraction device in claim 7, in which the flange is annular.

9. The retraction device as in claim 6 which the clamp is slidably mounted on the shaft.

10. The retraction device as in claim 9, in which the clamp includes a guide channel configured for receipt of the shaft, the channel guiding movement of the clamp along the shaft.

11. The retraction device as in claim 10, in which the guide channel is a bore.

12. The retraction device as in claim 11, in which the clamp includes a first member having a first channel and a second member having a counterpart second channel, the bore being formed by the mating of the first channel with the second channel.

13. The retraction device as in claim 12, in which the first member and second member are pivotally connected.

14. The retraction device as in claim 1, in which the shaft has a perimeter and the plurality of arms are arranged about the perimeter such that in the insertion profile a longitudinal edge of a first arm is in contact with a longitudinal edge of a neighbouring arm.

15. (canceled)

16. (canceled)

17. The retraction device as in any preceding claim, in which the plurality of arms are arranged about substantially all of said perimeter and extend therefrom.

18. (canceled)

19. (canceled)

20. The retraction device as in claim 1, in which at least one of the plurality of arms comprises a magnetic material providing at least one point for magnetic anchorage.

21. The retraction device as in claim 20, in which the magnetic material comprises a plurality of magnetic or electromagnetic elements, thereby providing multiple points for magnetic anchorage.

22. The retraction device as in claim 20, in which the magnetic material is embedded within the at least one arm in order to avoid direct contact with tissue.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A kit for use during a surgical procedure, the kit comprising a retraction device as in claim 1, and at least one of the group consisting of:
i) an access means through which the retraction device may be passed;
ii) a flexible attachment means configured to secure the retraction device to a rigid support means;
iii) a clamp configured for mounting on a shaft of the retraction device;
iv) a restraining means for attachment to at least one of the plurality of arms of the device, the restraining means being configured for restraining a tissue or organ;
v) a restraining means deployment tool.

47. (canceled)

48. (canceled)

49. The kit as in claim 46, in which the restraining means comprises a magnetic material, and in which the restraining means is magnetically attachable to at least one of a plurality of arms comprising a magnetic material.

50. (canceled)

51. (canceled)

52. (canceled)

53. (canceled)

54. (canceled)

55. (canceled)

56. (canceled)
